(19) United States
(12) Patent Application Publication
(10) Pub. No.: US 2023/0036976 A1
(43) Pub. Date: Feb. 2, 2023

(54) POLYMORPHISMS AS PREDICTORS OF TREATMENT RESPONSE AND OVERALL SURVIVAL OF METASTATIC COLORECTAL CANCER

(71) Applicant: AENORASIS COMMERCIAL COMPANY OF PHARMACEUTICAL AND MEDICAL PRODUCTS AND MACHINES SOCIETE ANONYME, Melissa (GR)

(72) Inventors: Grigorios Sivolapenko, Athens (GR); Apostolos Papachristos, Athens (GR)

(21) Appl. No.: 17/620,573

(22) PCT Filed: Jun. 19, 2020
(86) PCT No.: PCT/EP2020/067206

§ 371 Date: Dec. 17, 2021

(30) Foreign Application Priority Data
Jun. 21, 2019 (GR) 20190100271
Jul. 26, 2019 (GB) 1910731.7

(51) Int. Cl. C12Q 1/6886; A61K 39/395; A61K 31/7068; A61K 31/4745; A61K 31/282; A61P 35/04

(52) U.S. Cl. CPC C12Q 1/6886; A61K 39/3955; A61K 31/7068; A61K 31/4745; A61K 31/282; A61P 35/04; C12Q 2600/106; C12Q 2600/118; C12Q 2600/156; A61K 2039/505

(57) ABSTRACT

The present disclosure relates to methods for determining whether a subject with cancer, in particular metastatic colorectal cancer, is likely to respond to treatment and/or predicting overall survival with bevacizumab and fluoropyrimidine-based chemotherapy. The present disclosure also relates to methods of selecting a treatment of a subjects cancer and compounds for use in the treatment of the subjects cancer. The present disclosure also relates to kits that can be utilised in these methods.

### CLAIM OF PRIORITY

This application is a national phase application under 35 U.S.C. § 371 of PCT International Application number PCT/EP2020/067206, filed Jun. 19, 2020, which claims the benefit of Great Britain Patent Application Serial No. 1910731.7, filed on Jul. 26, 2019 and Greece Patent Application 20190100271 filed on Jun. 21, 2019. The entire contents of the foregoing are hereby incorporated by reference.

### DESCRIPTION OF INVENTION

### Field of the Invention

The present disclosure relates to methods for determining whether a subject with cancer, in particular metastatic colorectal cancer, is likely to respond to treatment and/or predicting overall survival with bevacizumab and fluoropyrimidine-based chemotherapy. The present disclosure also relates to methods of selecting a treatment of a subject's cancer and compounds for use in the treatment of the subject's cancer. The present disclosure also relates to kits that can be utilised in these methods.

### Background of the Invention

Colorectal cancer (CRC) is the second most commonly diagnosed cancer in women and the third most common in men. It is estimated that 20% of newly diagnosed patients have distant metastatic disease at the time of diagnosis. Major advances in the treatment of metastatic colorectal cancer (mCRC) over the last 15 years have greatly increased the average median overall survival for patients. However, there is great variability in the clinical outcome of patients.

Bevacizumab, a recombinant humanised IgG1 monoclonal antibody, is the first approved anti-angiogenetic agent for the treatment of mCRC. The mechanism of action of bevacizumab includes binding to circulating vascular endothelial growth factor A (VEGF-A) and blocking of VEGF-A binding to its receptors (VEGFR-1 and VEGFR-2) on the surface of endothelial cells, which results in the inhibition of tumor angiogenesis, growth, and metastases. Bevacizumab in combination with fluouropyrimidine-based chemotherapy is widely used as a first-line treatment for mCRC.

However, the clinical outcomes of treatment with bevacizumab mono- and combination therapies are highly variable. Similar to most monoclonal antibodies, bevacizumab exhibits complex and variable pharmacokinetic and pharmacodynamics characteristics. The variability in patient outcomes has been associated with several factors such as sex, body weight, tumor burden, binding to their molecular targets, and concomitantly administered chemotherapies.

Although bevacizumab is widely used in oncology, there is still a lack of validated predictive factors for treatment outcomes. Several clinical studies in patients with mCRC have noted a relationship between the concentration of biomarkers (e.g. lactate dehydrogenase, ICAM, E-selectin, endothelial nitric oxide synthase) and SNPs of genes involved in angiogenesis pathway and response to bevacizumab. There are no validated biomarkers currently available to guide patient selection for treatment with bevacizumab-based therapy.

The pathogenesis of mCRC involves the accumulation of genetic and epigenetic modifications within pathways that regulate proliferation, apoptosis and angiogenesis. RAS and BRAF genomic variants are of prognostic and predictive value in mCRC. KRAS genomic variants involving either codon 12 or 13 can be identified in 12-75% percent of CRCs and they have been independently associated with a worse prognosis. NRAS mutations are also associated with an inferior prognosis. Similarly, BRAF activating mutations, most occurring in codon 600 (V600E), are present in less than 10% of tumors and represent a strong negative prognostic marker.

There have been investigations into single nucleotide polymorphisms (SNPs) in genes involved in VEGF-dependent and independent angiogenesis pathways and other major intracellular signalling pathways involved in the pathogenesis of mCRC. In particular, VEGF-A and ICAM-1 gene polymorphisms have been identified that have been correlated to mCRC (see Zhang et al. Med. Sci. Monit. 2016; 22: 569-579 and Wether et al., Eur. J. Surg. Oncol., 2000; 26: 657-662; each of which are incorporated herein by reference). The role of VEGF-A SNPs on the therapeutic efficiency of bevacizumab-based therapy has been investigated in many tumors. For example, the genotype distribution of VEGF-A gene rs699947 polymorphism was analysed with respect to bevacizumab-based treatment for colorectal cancer. However, no significant correlation was determined for the VEGF-A gene rs699947 polymorphism with respect to response to treatment (see Cui et al., Oncotarget 2017, 8(62), pages 105472-104478; which is incorporated herein by reference).

A number of investigations have looked at PFS and OS. However, the investigations have been on a small scale and have provided mixed results. There have been no statistically significant findings that link VEGF-A and ICAM-1 gene polymorphisms to an improved clinical response and/or longer overall survival when the patient is treated with bevacizumab in combination with fluoropyrimidine-based chemotherapy.

There is a need to identify the population or populations that will benefit from bevacizumab treatment among mCRC patients, thus optimizing the management of the disease. In particular, there is a need for validated biomarkers to guide patient selection for treatment with bevacizumab in combination with fluoropyrimidine-based chemotherapy. There is a need for biomarkers that have been demonstrated to be statistically significant for increasing overall survival for bevacizumab in combination with fluoropyrimidine-based chemotherapy.

### SUMMARY OF THE INVENTION

Representative features of the present invention are set out in the following clauses, which stand alone or may be combined, in any combination, with one or more features disclosed in the text and/or drawings of the specification.

1. A method for determining whether a subject with metastatic colorectal cancer (mCRC) is likely to respond to the treatment and/or for predicting overall survival (OS) of the subject when treated with bevacizumab and fluoropyrimidine-based chemotherapy, comprising or consisting of the steps of: (a) identifying the presence or absence of the VEGF-A rs699947 polymorphism and/or ICAM-1 rs1799969 polymorphism in a sample obtained from the subject, or identifying a subject who has been identified as having the VEGF-A rs699947 polymorphism and/or ICAM-1 rs1799969 polymorphism; and

(b) indicating that the subject is more likely to respond to treatment with bevacizumab and fluoropyrimidine-based chemotherapy and/or likely to have a longer overall survival when treated with bevacizumab and fluoropyrimidine-based chemotherapy, if at least one of VEGF-A rs699947 polymorphism and ICAM-1 rs1799969 polymorphism is present.

2. A method according to clause 1, wherein the sample is a whole-blood, blood serum, peripheral blood leukocytes, or saliva.

3. A method according to any one of clauses 1 or 2, wherein the presence or absence of the allele is identified by PCR, PCR-RFLP, direct sequencing, TaqMan and/or next generation signalling.

4. The method of any one of clauses 1 to 3, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

5. The method of any one of clauses 1 to 3, wherein the fluoropyrimidine-based chemotherapy is capecitabine/irinotecan (BEV-CapIRI) or capecitabine/oxaliplatin (BEV-CapOX).

6. The method of any one of clauses 1 to 5, wherein the bevacizumab is administered as an intravenous infusion at a dose of 2.5 to 7.5 mg/kg once every 2 weeks in combination with the fluoropyrimidine-based chemotherapy; preferably the dose is 5 mg/kg.

7. The method of any one of clauses 1 to 5, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

8. The method according to any one of clauses 1 to 7, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

9. The method of any one of clauses 1 to 8, wherein the presence of the VEGF-A rs699947 polymorphism and/or ICAM-1 rs1799969 polymorphism in the sample obtained from the subject, or identifying a subject who has been identified as having the VEGF-A rs699947 polymorphism and/or ICAM-1 rs1799969 polymorphism, indicates that the subject is also more likely to respond to a maintenance treatment and/or likely to have a longer overall survival with a maintenance treatment.

10. The method according to any one of clauses 1 to 9, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

11. The method of any one of clauses 1 to 10, wherein there is no maintenance treatment.

12. The method of any one of clauses 9 to 10, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

12A. The method of any one of clauses 1 to 12, wherein a subject having the VEGF-A rs699947 polymorphism will have an OS of 33 to 71 months, 33.3 to 70.7 months, 45 to 65 months, 45 to 60 months, or 50 to 55 months, if treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, if treated with bevacizumab and fluoropyrimidine-based chemotherapy.

12B. The method of any one of clauses 1 to 12, wherein a subject having the ICAM-1 rs1799969 polymorphism will have an OS of 34 to 63 months, 34.5 to 62.8 months, 40 to 60 months, 45 to 55 months, or 50 to 55 months, if treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, if treated with bevacizumab and fluoropyrimidine-based chemotherapy.

13. A method of selecting a treatment for a subject's metastatic colorectal cancer, the method comprising, consisting essentially of, or consisting of the steps of:

(a) identifying the presence or absence of the VEGF-A rs699947 mutations and/or ICAM-1 rs1799969 mutations in a sample obtained from the subject, or identifying a subject who has been identified as having the VEGF-A rs699947 polymorphism and/or ICAM-1 rs1799969 polymorphism; and

(b) selecting the treatment comprising, consisting essentially of, or consisting of a bevacizumab and fluoropyrimidine-based chemotherapy if at least one of VEGF-A rs699947 mutations and ICAM-1 rs1799969 mutations is present.

14. The method according to clause 13, wherein the sample is a whole-blood, blood serum, peripheral blood leukocytes, or saliva.

15. The method according to any one of clauses 13 or 14, wherein the presence or absence of the allele is identified by PCR, PCR-RFLP, direct sequencing, TaqMan and/or next generation signalling.

16. The method of any one of clauses 13 to 15, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

17. The method of any one of clauses 13 to 15, wherein the fluoropyrimidine-based chemotherapy is capecitabine/irinotecan (BEV-CapIRI) and/or capecitabine/oxaliplatin (BEV-CapOX).

18. The method of any one of clauses 13 to 17, wherein the bevacizumab is administered as an intravenous infusion at a dose of 2.5 to 7.5 mg/kg once every 2 weeks in combination with the fluoropyrimidine-based chemotherapy; preferably the dose is 5 mg/kg.

19. The method of any one of clauses 13 to 17, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

20. The method according to any one of clauses 13 to 19, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

21. The method of any one of clauses 13 to 20, wherein the presence of the VEGF-A rs699947 polymorphism and/or ICAM-1 rs1799969 polymorphism in the sample obtained from the subject, or identifying a subject who has been identified as having the VEGF-A rs699947 polymorphism and/or ICAM-1 rs1799969 polymorphism, indicates that the subject is also more likely to respond to a maintenance treatment and/or likely to have a longer overall survival with a maintenance treatment.

22. The method according to any one of clauses 13 to 21, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

23. The method of any one of clauses 13 to 20, wherein there is no maintenance treatment.

24. The method of any one of clauses 21 to 22, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

24A. The method of any one of clauses 13 to 24, wherein a subject having the VEGF-A rs699947 polymorphism will have an OS of 33 to 71 months, 33.3 to 70.7 months, 45 to 65 months, 45 to 60 months, or 50 to 55 months, if treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, if treated with bevacizumab and fluoropyrimidine-based chemotherapy.

24B. The method of any one of clauses 13 to 24, wherein a subject having the ICAM-1 rs1799969 polymorphism will have an OS of 34 to 63 months, 34.5 to 62.8 months, 40 to 60 months, 45 to 55 months, or 50 to 55 months, if treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, if treated with bevacizumab and fluoropyrimidine-based chemotherapy.

25. A method of treating a subject's metastatic colorectal cancer, the method comprising consisting essentially of, or consisting of the steps of:

(a) identifying the presence or absence of the VEGF-A rs699947 polymorphism and/or ICAM-1 rs1799969 polymorphism in a sample obtained from the subject, or identifying a subject who has been identified as having the VEGF-A rs699947 polymorphism and/or ICAM-1 rs1799969 polymorphism; and

(b) administering bevacizumab and fluoropyrimidine-based chemotherapy, if at least one of VEGF-A rs699947 polymorphism and ICAM-1 rs1799969 polymorphism is present.

26. A method according to clause 25, wherein the sample is a whole-blood, blood serum, peripheral blood leukocytes, or saliva.

27. A method according to any one of clauses 25 or 26, wherein the presence or absence of the allele is identified by PCR, PCR-RFLP, direct sequencing, TaqMan and/or next generation signalling.

28. The method of any one of clauses 25 to 27, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

29. The method of any one of clauses 25 to 27, wherein the fluoropyrimidine-based chemotherapy is capecitabine/irinotecan (BEV-CapIRI) and/or capecitabine/oxaliplatin (BEV-CapOX).

30. The method of any one of clauses 25 to 29, wherein the bevacizumab is administered as an intravenous infusion at a dose of 2.5 to 7.5 mg/kg once every 2 weeks in combination with the fluoropyrimidine-based chemotherapy; preferably the dose is 5 mg/kg.

31. The method of any one of clauses 25 to 29, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

32. The method according to any one of clauses 25 to 31, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

33. The method of any one of clauses 25 to 32, wherein the presence of the VEGF-A rs699947 polymorphism and/or ICAM-1 rs1799969 polymorphism in the sample obtained from the subject, or identifying a subject who has been identified as having the VEGF-A rs699947 polymorphism and/or ICAM-1 rs1799969 polymorphism, indicates that the subject is also more likely to respond to a maintenance treatment and/or likely to have a longer overall survival with a maintenance treatment.

34. The method according to any one of clauses 25 to 33, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

35. The method of any one of clauses 25 to 32, wherein there is no maintenance treatment.

36. The method of any one of clauses 33 to 34, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

36A. The method of any one of clauses 25 to 36, wherein a subject having the VEGF-A rs699947 polymorphism will have an OS of 33 to 71 months, 33.3 to 70.7 months, 45 to 65 months, 45 to 60 months, or 50 to 55 months, if treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, if treated with bevacizumab and fluoropyrimidine-based chemotherapy.

36B. The method of any one of clauses 25 to 36, wherein a subject having the ICAM-1 rs1799969 polymorphism will have an OS of 34 to 63 months, 34.5 to 62.8 months, 40 to 60 months, 45 to 55 months, or 50 to 55 months, if treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, if treated with bevacizumab and fluoropyrimidine-based chemotherapy.

37. A combination of bevacizumab and a fluoropyrimidine-based chemotherapy, for use in a method of treating a subject's metastatic colorectal cancer, the method comprising the steps of:

(a) identifying the presence or absence of the VEGF-A rs699947 polymorphism and/or ICAM-1 rs1799969 polymorphism in a sample obtained from the subject, or identifying a subject who has been identified as having the VEGF-A rs699947 polymorphism and/or ICAM-1 rs1799969 polymorphism; and

(b) administering bevacizumab and fluoropyrimidine-based chemotherapy, if at least one of VEGF-A rs699947 polymorphism and ICAM-1 rs1799969 polymorphism is present.

38. A method according to clause 37, wherein the sample is a whole-blood, blood serum, peripheral blood leukocytes, or saliva.

39. A method according to any one of clauses 37 or 38, wherein the presence or absence of the allele is identified by PCR, PCR-RFLP, direct sequencing, TaqMan and/or next generation signalling.

40. The method of any one of clauses 37 to 39, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

41. The method of any one of clauses 37 to 39, wherein the fluoropyrimidine-based chemotherapy is capecitabine/irinotecan (BEV-CapIRI) and/or capecitabine/oxaliplatin (BEV-CapOX).

42. The method of any one of clauses 37 to 41, wherein the bevacizumab is administered as an intravenous infusion at a dose of 2.5 to 7.5 mg/kg once every 2 weeks in combination with the fluoropyrimidine-based chemotherapy; preferably the dose is 5mg/kg.

43. The method of any one of clauses 37 to 41, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

44. The method according to any one of clauses 37 to 43, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

45. The method of any one of clauses 37 to 44, wherein the presence of the VEGF-A rs699947 polymorphism and/or ICAM-1 rs1799969 polymorphism in the sample obtained from the subject, or identifying a subject who has been identified as having the VEGF-A rs699947 polymorphism and/or ICAM-1 rs1799969 polymorphism, indicates that the subject is also more likely to respond to a maintenance treatment and/or likely to have a longer overall survival with a maintenance treatment.

46. The method according to any one of clauses 37 to 45, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

47. The method of any one of clauses 37 to 44, wherein there is no maintenance treatment.

48. The method of any one of clauses 45 to 46, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

48A. The method of any one of clauses 37 to 48, wherein a subject having the VEGF-A rs699947 polymorphism will have an OS of 33 to 71 months, 33.3 to 70.7 months, 45 to 65 months, 45 to 60 months, or 50 to 55 months, if treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, if treated with bevacizumab and fluoropyrimidine-based chemotherapy.

48B. The method of any one of clauses 37 to 48, wherein a subject having the ICAM-1 rs1799969 polymorphism will have an OS of 34 to 63 months, 34.5 to 62.8 months, 40 to 60 months, 45 to 55 months, or 50 to 55 months, if treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, if treated with bevacizumab and fluoropyrimidine-based chemotherapy.

49. A method of treating metastatic colorectal cancer in a subject having the VEGF-A rs699947 polymorphism and/or ICAM-1 rs1799969 polymorphism, the method comprising administering bevacizumab and fluoropyrimidine-based chemotherapy.

50. A method according to clause 49, wherein the presence or absence of the VEGF-A rs699947 polymorphism and/or ICAM-1 rs1799969 polymorphism can be identified using a sample obtained from the subject, and the sample is a whole-blood, blood serum, peripheral blood leukocytes, or saliva.

51. A method according to any one of clauses 49 or 50, wherein the presence or absence of the allele is identified by PCR, PCR-RFLP, direct sequencing, TaqMan and/or next generation signalling.

51A. The method according to any one of clauses 49 or 50, wherein the subject has been identified as having the VEGF-A rs699947 polymorphism and/or ICAM-1 rs1799969 polymorphism.

52. The method of any one of clauses 49 to 51A, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

53. The method of any one of clauses 49 to 51A, wherein the fluoropyrimidine-based chemotherapy is capecitabine/irinotecan (BEV-CapIRI) and/or capecitabine/oxaliplatin (BEV-CapOX).

54. The method of any one of clauses 49 to 53, wherein the bevacizumab is administered as an intravenous infusion at a dose of 2.5 to 7.5 mg/kg once every 2 weeks in combination with the fluoropyrimidine-based chemotherapy; preferably the dose is 5 mg/kg.

55. The method of any one of clauses 49 to 53, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

56. The method according to any one of clauses 49 to 55, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

57. The method of any one of clauses 49 to 56, wherein the presence of the VEGF-A rs699947 polymorphism and/or ICAM-1 rs1799969 polymorphism in the sample obtained from the subject, or when the subject has been identified as having the VEGF-A rs699947 polymorphism and/or ICAM-1 rs1799969 polymorphism, indicates that the subject is also more likely to respond to a maintenance treatment and/or likely to have a longer overall survival with a maintenance treatment.

58. The method according to any one of clauses 49 to 57, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

59. The method of any one of clauses 49 to 56, wherein there is no maintenance treatment.

60. The method of any one of clauses 57 to 58, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

60A. The method of any one of clauses 49 to 60, wherein a subject having the VEGF-A rs699947 polymorphism will have an OS of 33 to 71 months, 33.3 to 70.7 months, 45 to 65 months, 45 to 60 months, or 50 to 55 months, when treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, when treated with bevacizumab and fluoropyrimidine-based chemotherapy.

60B. The method of any one of clauses 49 to 60, wherein a subject having the ICAM-1 rs1799969 polymorphism will have an OS of 34 to 63 months, 34.5 to 62.8 months, 40 to 60 months, 45 to 55 months, or 50 to 55 months, when treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, when treated with bevacizumab and fluoropyrimidine-based chemotherapy.

61. A combination of bevacizumab and a fluoropyrimidine-based chemotherapy, for use in a method of treating a subject's metastatic colorectal cancer, wherein the subject has the VEGF-A rs699947 polymorphism and/or ICAM-1 rs1799969 polymorphism.

62. A method according to clause 61, wherein the presence or absence of the VEGF-A rs699947 polymorphism and/or ICAM-1 rs1799969 polymorphism can be identified using a sample obtained from the subject, and the sample is a whole-blood, blood serum, peripheral blood leukocytes, or saliva.

63. A method according to any one of clauses 61 or 62, wherein the presence or absence of the allele is identified by PCR, PCR-RFLP, direct sequencing, TaqMan and/or next generation signalling.

63A. The method according to any one of clauses 49 or 50, wherein the subject has been identified as having the VEGF-A rs699947 polymorphism and/or ICAM-1 rs1799969 polymorphism.

64. The method of any one of clauses 61 to 63A, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

65. The method of any one of clauses 61 to 63A, wherein the fluoropyrimidine-based chemotherapy is capecitabine/irinotecan (BEV-CapIRI) and/or capecitabine/oxaliplatin (BEV-CapOX).

66. The method of any one of clauses 61 to 65, wherein the bevacizumab is administered as an intravenous infusion at a dose of 2.5 to 7.5 mg/kg once every 2 weeks in combination with the fluoropyrimidine-based chemotherapy; preferably the dose is 5 mg/kg.

67. The method of any one of clauses 61 to 65, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

68. The method according to any one of clauses 61 to 67, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

69. The method of any one of clauses 61 to 68, wherein the presence of the VEGF-A rs699947 polymorphism and/or ICAM-1 rs1799969 polymorphism in the sample obtained from the subject, or when the subject has been identified as having the VEGF-A rs699947 polymorphism and/or ICAM-1 rs1799969 polymorphism, indicates that the subject is also more likely to respond to a maintenance treatment and/or likely to have a longer overall survival with a maintenance treatment.

70. The method according to any one of clauses 61 to 69, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

71. The method of any one of clauses 61 to 68, wherein there is no maintenance treatment.

72. The method of any one of clauses 69 to 70, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

72A. The method of any one of clauses 61 to 72, wherein a subject having the VEGF-A rs699947 polymorphism will have an OS of 33 to 71 months, 33.3 to 70.7 months, 45 to 65 months, 45 to 60 months, or 50 to 55 months, when treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, when treated with bevacizumab and fluoropyrimidine-based chemotherapy.

72B. The method of any one of clauses 61 to 72, wherein a subject having the ICAM-1 rs1799969 polymorphism will have an OS of 34 to 63 months, 34.5 to 62.8 months, 40 to 60 months, 45 to 55 months, or 50 to 55 months, when treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, when treated with bevacizumab and fluoropyrimidine-based chemotherapy.

73. A method of treating cancer a subject with metastatic colorectal cancer (mCRC) comprising or consisting of the steps of:

administering bevacizumab and fluoropyrimidine-based chemotherapy to a subject; and

identifying the presence or absence of the VEGF-A rs699947 polymorphism and/or ICAM-1 rs1799969 polymorphism in a sample obtained from the subject, or identifying a subject who has been identified as having the VEGF-A rs699947 polymorphism and/or ICAM-1 rs1799969 polymorphism.

74. A method according to clause 73, wherein the presence or absence of the VEGF-A rs699947 polymorphism and/or ICAM-1 rs1799969 polymorphism can be identified using a sample obtained from the subject, and the sample is a whole-blood, blood serum, peripheral blood leukocytes, or saliva.

75. A method according to any one of clauses 73 or 74, wherein the presence or absence of the allele is identified by PCR, PCR-RFLP, direct sequencing, TaqMan and/or next generation signalling.

75A. The method according to any one of clauses 73 or 75, wherein the subject has been identified as having the VEGF-A rs699947 polymorphism and/or ICAM-1 rs1799969 polymorphism.

75B. The method of any one of clauses 73 to 75A, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

76. The method of any one of clauses 73 to 75A, wherein the fluoropyrimidine-based chemotherapy is capecitabine/irinotecan (BEV-CapIRI) and/or capecitabine/oxaliplatin (BEV-CapOX).

77. The method of any one of clauses 73 to 76, wherein the bevacizumab is administered as an intravenous infusion at a dose of 2.5 to 7.5 mg/kg once every 2 weeks in combination with the fluoropyrimidine-based chemotherapy; preferably the dose is 5 mg/kg.

78. The method of any one of clauses 73 to 76, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

79. The method according to any one of clauses 73 to 78, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

80. The method according to any one of clauses 73 to 79, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

81. The method of any one of clauses 73 to 79, wherein there is no maintenance treatment.

82. The method of any one of clauses 80 to 81, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

82A. The method of any one of clauses 73 to 82, wherein a subject having the VEGF-A rs699947 polymorphism will have an OS of 33 to 71 months, 33.3 to 70.7 months, 45 to 65 months, 45 to 60 months, or 50 to 55 months, when treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, when treated with bevacizumab and fluoropyrimidine-based chemotherapy.

82B. The method of any one of clauses 49 to 60, wherein a subject having the ICAM-1 rs1799969 polymorphism will have an OS of 34 to 63 months, 34.5 to 62.8 months, 40 to 60 months, 45 to 55 months, or 50 to 55 months, when treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, when treated with bevacizumab and fluoropyrimidine-based chemotherapy.

83. A method of identifying identifying the presence or absence of the VEGF-A rs699947 polymorphism and/or ICAM-1 rs1799969 polymorphism in a subject, the method comprising the steps of:
(a) Collecting a whole blood sample,
(b) Separating the blood and isolating the serum sample,
(c) Extracting the genomic DNA from blood leukocytes,
(d) Measuring the DNA concentration,
(e) Analysing the genomic variant by PCR and purifying the PCR products,
(f) Carrying our DNA sequence analysis on the purified PRC products; or
(g) adding a label that binds to the VEGF-A rs699947 polymorphism and/or ICAM-1 rs1799969 polymorphism and detecting the bound label.

84. A method for treating metastatic colorectal cancer in a subject, the method comprising administering a combination of bevacizumab and a fluoropyrimidine-based chemotherapy to a subject known to have the ICAM-1 rs1799969 G/A polymorphism.

85. A method for administering a combination of bevacizumab and a fluoropyrimidine-based chemotherapy to a subject having metastatic colorectal cancer, the method comprising identifying the presence or absence of the ICAM-1 rs1799969 G/A polymorphism in a sample obtained from the subject and administering the combination of bevacizumab and a fluoropyrimidine-based chemotherapy to the subject having the ICAM-1 rs1799969 G/A polymorphism.

86. A method for treating metastatic colorectal cancer in a subject, the method comprising administering a combination of bevacizumab and a fluoropyrimidine-based chemotherapy to a subject known to have the VEGF-A rs699947 A/A polymorphism.

87. A method for administering a combination of bevacizumab and a fluoropyrimidine-based chemotherapy to a subject having metastatic colorectal cancer, the method comprising identifying the presence or absence of the VEGF-A rs699947 A/A polymorphism in a sample obtained from the subject and administering the combination of bevacizumab and a fluoropyrimidine-based chemotherapy to the subject having the VEGF-A rs699947 A/A polymorphism.

88. A method according to any one of clauses 84 to 87, wherein the combination of bevacizumab and a fluoropyrimidine-based chemotherapy is administered as first-line treatment.

89. A method according to any one of clauses 84 to 88, wherein the step of identifying comprises detecting the polymorphism by a method comprising one or more of PCR, PCR-RFLP, direct sequencing, TaqMan and/or next generation sequencing.

90. A method according to any one of clauses 84 to 89, wherein the sample is a whole-blood, blood serum, peripheral blood leukocytes, or saliva.

91. The method of any one of clauses 84 to 90, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

92. The method of any one of clauses 84 to 90, wherein the fluoropyrimidine-based chemotherapy is capecitabine/irinotecan (BEV-CapIRI) and/or capecitabine/oxaliplatin (BEV-CapOX).

93. The method of any one of clauses 84 to 92, wherein the bevacizumab is administered as an intravenous infusion at a dose of 2.5 to 7.5 mg/kg once every 2 weeks in combination with the fluoropyrimidine-based chemotherapy; preferably the dose is 5 mg/kg.

94. The method of any one of clauses 84 to 92, wherein the bevacizumab is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

95. The method according to any one of clauses 84 to 94, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

96. The method according to any one of clauses 84 to 95, wherein the patient has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 maintenance treatment cycles.

97. The method of any one of clauses 84 to 95, wherein there is no maintenance treatment.

98. The method of clause 96, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

98A. The method of any one of clauses 84 to 98, wherein a subject having the VEGF-A rs699947 polymorphism will have an OS of 33 to 71 months, 33.3 to 70.7 months, 45 to 65 months, 45 to 60 months, or 50 to 55 months, when treated with bevacizumab and fluoropyrimidine-based chemotherapy; or a subject having the VEGF-A rs699947 polymorphism will have an OS of 33, 44, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, when treated with bevacizumab and fluoropyrimidine-based chemotherapy.

98B. The method of any one of clauses 84 to 98, wherein a subject having the ICAM-1 rs1799969 polymorphism will have an OS of 34 to 63 months, 34.5 to 62.8 months, 40 to 60 months, 45 to 55 months, or 50 to 55 months, when treated with bevacizumab and fluoropyrimidine-based chemotherapy; or a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, when treated with bevacizumab and fluoropyrimidine-based chemotherapy.

### BRIEF DESCRIPTION OF THE DRAWINGS

Embodiments of the invention are described below with reference to the accompanying drawings, in which:

FIG. 1. shows the OS according to VEGF-A rs699947 polymorphism. Median OS was 52 months in A/A carriers vs. 18.1 months in C/C carriers (p=0.043).

FIG. 2. shows the OS according to ICAM-1 rs1799969 polymorphism. Median OS was 48.7 months in G/A carriers vs. 29.1 months in G/G carriers (p=0.036).

FIG. 3. shows the PFS according to VEGF-A rs699947 polymorphism. Median PFS was 31.1 months in A/A carriers vs. 10.1 months in C/C carriers (P=0.006).

### DETAILED DESCRIPTION OF THE INVENTION

The following description and examples illustrate various embodiments of the present disclosure in detail. Those of skill in the art will recognize that there are numerous variations and modifications of this disclosure that are encompassed by its scope. Accordingly, the description of the disclosed embodiments should not be deemed to limit the scope of the present disclosure.

Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

“Bevacizumab” refers to the compound Avastin®, a recombinant humanized monoclonal IgGI antibody that binds to and inhibits the biologic activity of human vascular endothelial growth factor (VEGF-A). The interaction of VEGF with its receptors leads to endothelial cell proliferation and new blood vessel formation in in vitro models of angiogenesis. Bevacizumab binds to circulating VEGF-A and blocks VEFG-A from binding to is receptors (VEGFR-1 and VEGFR-2) on the surface of endothelial cells, which results in the inhibition of tumour angiogenesis, growth and metastases. “Bevacizumab” encompass all corresponding anti-VEGF antibodies or anti-VEGF antibody fragments that fulfil the requirements necessary for obtaining a marketing authorization as an identical or biosimilar product to the reference medicine/reference product bevacizumab (Avastin®) by a regulatory authority such as the European Medicines Agency (EMA), the United States Food and Drug Administration (FDA), or the like. See the EMA's “Guideline on similar biological medicinal products containing monoclonal antibodies—non-clinical and clinical issues” (https://www.ema.europa.eu/en/documents/scientific-guideline/guideline-similar-biological-medicinal-products-containing-monoclonal-antibodies-non-clinical_en.pdf). See the FDA's “Scientific Considerations in Demonstrating Biosimilarity to a Reference Product” (https://www.fda.gov/media/82647/download). Biosimilars have been approved as a biosimilar medicine to the reference medicine/reference product bevacizumab (Avastin®). For example but not limited to: Mvasi (ABP-215) or Mvasi (bevacizumab-awwb) (Amgen) and Zirabev (Pfizer). Mvasi has been approved as a biosimilar of reference medicine/reference product bevacizumab (Avastin®) by the EMA and FDA. Zirabev has been approved as a biosimilar the reference medicine/reference product bevacizumab (Avastin®) by the EMA. These biosimilars bind to and inhibit the biologic activity of human vascular endothelial growth factor (VEGF-A). Biosimilars are approved according to the same standards of pharmaceutical quality, safety and efficacy that apply to all biological medicines.

A number of biosimilars are also being developed, such as but not limited to BCD-021 (Biocad, Russia); FKB238 (AstraZeneca/Fujifilm Kyowa Kirin Biologics, USA/Japan); BCD500 (BIOCND, South Korea); Krabeva (Biocon, India*); BI 695502 (Boehringer Ingelheim, Germany); CT-P16 (Celltrion, South Korea); CHS-5217 (Coherus, USA); DRZ_BZ (Dr Reddy's Laboratories, India); Cizumab (Hetero (Lupin), India); Bevax (mAbxience, Spain (Argentina)); ONS-1045 (OncobiologicsNiropro, USA); PF-06439535 (Pfizer, USA); HD204 (Prestige Biopharma, Singapore); Bevacirel (Reliance Life Sciences/Lupin, India); and SB8 (Samsung Bioepis (Biogen/Samsung)/Merck, South Korea/USA); these compounds will be biosimilars in accordance with the invention if they are approved as a biosimilar the bevacizumab (Avastin®) bevacizumab (Avastin®) by an appropriate regulatory authority such as the European Medicines Agency (EMA), the United States Food and Drug Administration (FDA), or the like.

Bevacizumab treatment can be in the form of monotherapy and/or in combination with chemotherapy, in particular fluoropyrimidine-based chemotherapy as described herein. For example, the bevacizumab treatment can be a first-line treatment of bevacizumab in combination with a fluoropyrimidine-based chemotherapy, and followed by a maintenance treatment of bevacizumab monotherapy.

As used herein, “metastatic colorectal cancer (mCRC)” refers to subjects with metastatic carcinoma of the colon or rectum. In mCRC, the cancer has spread from the colon or rectum to distant organs and tissues. Colorectal cancer most often spreads to the liver, but it can also spread to other places such as the lungs, bones, brain, spinal cord, peritoneum (the lining of the abdominal cavity), or to distant lymph nodes.

As used herein, “VEGF-A” is part of the VEGF family. Vascular endothelial growth factor A (VEGF-A) is a protein that is encoded by the VEGFA gene. VEGF-A binds with high affinity to receptors VEGFR-1 and VEGFR-1 and promotes angiogenic signals. VEGF-A is considered the major activator of angiogenesis. It acts selectively on vascular endothelial cells, stimulating both normal and abnormal angiogenesis.

As used herein, “ICAM-1” is known to be a member of the immunoglobulin gene superfamily of adhesion molecules. Intercellular adhesion molecule-1 (ICAM-1) is a cell surface glycoprotein which is typically expressed on endothelial cells and cells of the immune system. It binds to integrins of type CD11a/CD18, or CD11b/CD18. ICAM-1 expression is closely associated with cancer metastasis.

As used herein, “single nucleotide polymorphisms (SNPs)” refers to a substitution of a single nucleotide that occurs at a specific position in the genome. For example, at a specific base position in the human genome, the C nucleotide may appear in most individuals, but in a minority of individuals, the position is occupied by an A. This means that there is a SNP at this specific position, and the two possible nucleotide variations—C or A—are said to be alleles for this position. The VEGF-A rs699947 polymorphism and the ICAM-1 rs1799969 polymorphism are SNPs.

As used herein “VEGF-A rs699947 polymorphism”, “VEGF-A rs699947”, “VEGF-A rs699947 gene polymorphism” and “VEGF-A rs699947 mutant” refer to a single nucleotide polymorphism of the VEGF-A gene at position 2578. A subject may be a carrier of A/A allele or C/C allele. “VEGF-A rs699947 allele A/A” refers to a subject carrying the allele A/A at position 2578 of the VEGF-A gene, which is an SNP and referred to as a VEGF-A polymorphism. “VEGF-A rs699947 allele C/C” refers to a subject carrying the allele C/C at position 2578 of the VEGF-A gene, which is the wild type version of the gene.

As used herein, “ICAM-1 rs1799969 polymorphism”, “ICAM-1 rs1799969”, “ICAM-1 rs1799969 gene polymorphism” and “ICAM-1 rs1799969 mutant” refer to a single nucleotide polymorphism of the ICAM-1 gene at position 241. A subject may be a carrier of the G/G allele or G/A allele. “ICAM-1 rs1799969 allele G/A” refers to a subject carrying the allele G/A at position 241 of the ICAM-1 gene, which is an SNP and referred to as an ICAM-1 polymorphism. “ICAM-1 rs1799969 allele G/G” refers to a subject carrying the allele G/G at position 241of the ICAM-1 gene, which is the wild type version of the gene.

As used herein, “fluoropyrimidine-based chemotherapy” refers to a group of substances used to treat cancer. A fluoropyrimidine is a type of antimetabolite; for example, capecitabine, floxuridine, and fluorouracil (5-FU). In some fluoropyrimidene-based chemotherapies, the fluoropyrimidine is administered with oxaliplatin and/or irinotecan. For example, 5-fluorouracil/leucovorin/irinotecan (FOLFIRI), 5-fluorouracil/leucovorin/oxaliplatin (FOLFOX), capecitabine/irinotecan (CapIRI) or capecitabine/oxaliplatin (CapOX), and 5-fluorouracil/leucovorin/oxaliplatin/irinotecan (FOLFOXIRI).

As used herein “progression free survival” or “PFS” refers to the length of time during and after the treatment of a disease, such as cancer, that a patient lives with the disease but it does not get worse. In a clinical trial, measuring the progression-free survival is one way to see how well a new treatment works. OS is a different way to see how well a new treatment works. Progression free surivival is not linked to OS. It is not possible to predict the outcome of OS based on PFS; there is no association between the two end points.

As used herein “likely to respond to treatment” refers to an increased probability of responding to treatment with bevacizumab in combination with fluoropyrimidine-based chemotherapy relative to a subject or a group of subjects not carrying the relevant polymorphism. For example, subjects carrying the VEGF-A rs699947 polymorphism, who are more likely to respond to treatment with bevacizumab and fluoropyrimidine-based chemotherapy, have an increased probability of responding to the treatment than a subject or group of subjects carrying the wild type VEGF-A rs699947. For example, subjects carrying the ICAM-1 rs1799969 polymorphism, who are more likely to respond to treatment with bevacizumab and fluoropyrimidine-based chemotherapy, have an increased probability of responding to the treatment than a subject or group of subjects carrying the wild type ICAM-1 rs1799969. A subject responds to treatment if the subject's life expectancy is extended beyond the life expectancy predicted if no treatment is administered. A subject responds to treatment if the subject has increased overall survival compared to the overall survival if no treatment is administered. The RECIST criteria can be used to determine if a patient responds to treatment and/or clinical assessment.

As used herein “overall survival” or “OS” refers to the time as measured from the start of the treatment to death or the end of the treatment or a change of treatment. In a clinical trial, measuring the overall survival is one way to see how well a new treatment works. Overall survival refers to the probability as, for example, a probability when represented in a Kaplan-Meier plot of being alive at a particular time between the start of the treatment and death. Survival analysis was performed with cox regression and Kaplan Meier plots, which are the most widely used statistical tools for survival data analysis. In addition, null hypothesis were checked with ling-rank test to ensure significance. These statistical tools are well known and widely used by those skilled in the art.

As used herein “RECIST” refers to “Response Evaluation Criteria in Solid Tumours” and is a set of published rules that define when cancer patients respond during treatments. The response was evaluated according to RECIST criteria version 1.1, which was published Eisenhauer et al., European Journal of Cancer, 45, 2009; 228-247; which is incorporated herein by reference. The RECIST criterion is standard methodology for those skilled in the art.

As used herein “ECOG performance status” is a standard criteria for measuring how a disease impacts a patient's daily living abilities (known to physicians and researchers as a patient's performance status). The ECOG Scale of Performance Status describes a patient's level of functioning in terms of their ability to care for themselves, daily activity, and physical ability (walking, working, etc.). The numbering scale is one way to define the population of patients to be studied in the trial, so that it can be uniformly reproduced among physicians who enrol patients. The scale was developed by the Eastern Cooperative Oncology Group (ECOG), now part of the ECOG-ACRIN Cancer Research Group, and published in 1982. It is displayed below:

| GRADE | ECOG PERFORMANCE STATUS |
|---|---|
| 0 | Fully active, able to carry on all pre-disease |
| | performance without restriction |
| 1 | Restricted in physically strenuous activity but |
| | ambulatory and able to carry out work of a light |
| | or sedentary nature, e.g., light house work, office work |
| 2 | Ambulatory and capable of all selfcare but unable to |
| | carry out any work activities; up and about more than |
| | 50% of waking hours |
| 3 | Capable of only limited selfcare; confined to bed or |
| | chair more than 50% of waking hours |
| 4 | Completely disabled; cannot carry on any selfcare; totally |
| | confined to bed or chair |
| 5 | Dead |

As used herein, “sample” refers to any sample that can provide genomic DNA for analysis to determine the absence or presence of the VEGF-A rs699947 polymorphism and/or the ICAM-1 rs1799969 polymorphism. These samples are well known in the art and include, but are not limited to: whole blood, blood serum, peripheral blood leukocytes, or saliva.

As used herein, “associated with a prolonged OS” or “associated with a longer OS” means that the polymorphism is found within patients who are more likely to respond to and/or have a longer overall survival with bevacizumab with fluoropyrimidine-based chemotherapy treatment than patents who have the different polymorphism. For example, patients carrying the polymorphism ICAM-1 rs1799969 polymorphism (G/A allele) are more likely to respond to bevacizumab with fluoropyrimidine-based chemotherapy treatment than patents carrying the wild-type ICAM-1 rs1799969 (G/G allele). For example, patients carrying the polymorphism VEGF-A rs699947 polymorphism (A/A allele) are more likely to respond to bevacizumab with fluoropyrimidine-based chemotherapy treatment than patents carrying the wild-type ICAM-1 rs1799969 (C/C allele). The association has statistical significance.

As referred to herein, “statistical significant”, “statistically significant”, “significant” or “significantly” refers to the likelihood that a relationship between two or more variables is caused by something other than chance. Statistical hypothesis testing is used to determine whether the result of a data set is statistically significant. This test provides a p-value, representing the probability that random chance could explain the result. A p-value of 5% or lower (p<0.05) is considered to be statistically significant. For example, the

VEGF-A rs699947 polymorphism (allele A/A) has been significantly associated with prolonged OS (p=0.043, i.e. less than 5%) relative to wild-type VEGF-A rs699947 (allele C/C). The ICAM-1 rs1799969 polymorphism (allele G/A) has been significantly associated with prolonged OS (p=0.036, i.e. less than 5%) relative to the wild-type ICAM-1 rs1799969 (allele G/G).

As used herein, “log-rank test” refers to a hypothesis test to compare the survival distributions of two samples. It is widely used in clinical trials to establish the efficacy of a new treatment in comparison with a control treatment when the measurement is the time to event (such as the time from initial treatment to death). This test is well known to the skilled person and described in Bland et al., BMJ 2004; 328:1073; which is incorporated herein by reference. Log-rank tests is more sophisticated and can take into account non-normality of study population compared to other hypothesis tests, such as ANOVA that provides only a brief analysis of raw data

As used herein, “Cox regression” refers to a method for investigating the effect of several variables upon the time a specified event takes to happen. In the context of an outcome such as death this is known as Cox regression for survival analysis. To address any feasibility limitations in recruitment, the inventors calculated the effect size that this study could capture for the pre-determined number of patients. At a power level of 80% and alpha set as 0.05, for 46 patients with 60 months of follow up and 1 year of recruitment we detected at least 1.5 years of difference in the median of PFS and OS between different polymorphisms.

The term “subject” refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. The subject can be, for example, a child, such as an adolescent, or an adult. Preferably the subject is an adult.

The term “treatment” refers to any treatment of a pathologic condition in a subject, such as a mammal, particularly a human, and includes: (i) preventing and/or reducing the risk of a pathologic condition from occurring in a subject which may be predisposed to the condition but has not yet been diagnosed with the condition and, accordingly, the treatment constitutes prophylactic treatment for the disease condition; (ii) inhibiting and/or reducing the speed of development of the pathologic condition, e.g., arresting its development; (iii) relieving the pathologic condition, e.g., causing regression of the pathologic condition; or (iv) relieving the conditions mediated by the pathologic condition and/or symptoms of the pathologic condition. Treatment to subjects who have previously and/or are currently, and/or are about to receive a cancer therapy are contemplated herein.

The term “therapeutically effective amount” refers to that amount of a compound, such as bevacizumab and/or fluoropyrimidine-based chemotherapy, that is sufficient to effect treatment, when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “consisting essentially of” means that specific further components can be present, namely those not materially affecting the essential characteristics of the compound or composition.

Where a range of values is provided, it is understood that the upper and lower limit, and each intervening value between the upper and lower limit of the range is encompassed within the embodiments.

The present invention investigated the role of SNPs in the VEGF-A and ICAM-1 angiogenesis genes, as well as, in the KRAS, NRAS and BRAF genes, in order to predict clinical outcome and tumor response in mCRC patients treated with bevacizumab in combination with fluoropyrimidine based chemotherapy. Genes and polymorphisms known to regulate proliferation, apoptosis and to modulate VEGF-induced or non-VEGF-induced angiogenesis were selected.

The present invention is based on the finding that the SNPs VEGF-A rs699947 polymorphism (allele A/A) and ICAM-1 rs1799969 polymorphism (allele G/A) are predictors of likelihood to respond to bevacizumab in combination with fluoropyrimidine-based chemotherapy in patients with mCRC. The VEGF-A rs699947 polymorphism has been significantly associated with prolonged OS (p=0.043) relative to wild-type VEGF-A rs699947. The ICAM-1 rs1799969 polymorphism has been significantly associated with prolonged OS (p=0.036) relative to the ICAM-1 rs1799969 allele G/G.

The statistically significant finding that subjects carrying the VEGF-A rs699947 polymorphism and/or ICAM-1 rs1799969 polymorphism have a prolonged OS compared to a subject not having these polymorphisms (i.e. carrying the wild-type alleles) provides for the identification of a new group of patients who will gain a long term benefit from treatment with bevacizumab in combination with fluoropyrimidine-based chemotherapy.

This finding means those subjects carrying the VEGF-A rs699947 polymorphism and/or ICAM-1 rs1799969 polymorphism are likely to respond and/or have a long overall survival (OS) when treated with bevacizumab and fluoropyrimidine-based chemotherapy.

This finding allows a physician to select a treatment for the mCRC subject's based on the present or absence of SNPs VEGF-A rs699947 polymorphism and ICAM-1 rs1799969 polymorphism in the subject's genomic DNA. This provides a financial benefit for insurance companies or healthcare systems, such as the NHS. For example, in the UK, bevacizumab is not approved due to lack of cost-effectiveness, because only a small number of patients benefit from the addition of bevacizumab to their chemotherapy. However, this study indicates that about 27% of the patients having the VEGF-A rs699947 polymorphism and/or ICAM-1 rs1799969 polymorphism will benefit from the addition of bevacizumab in their chemotherapy.

This finding also allows a physician to administer bevacizumab in combination with fluoropyrimidine-based chemotherapy to treat mCRC, based on the present or absence of SNPs VEGF-A rs699947 polymorphism and ICAM-1 rs1799969 polymorphism in the subject's genomic DNA.

The invention provides a method for determining whether a subject with mCRC is likely to respond to the treatment and/or for predicting overall survival (OS) of the subject when treated with bevacizumab and fluoropyrimidine-based chemotherapy, comprising the steps of (a) identifying the presence or absence of the VEGF-A rs699947 polymorphism and/or ICAM-1 rs1799969 polymorphism in a sample obtained from the subject, or identifying a subject who has been identified as having the VEGF-A rs699947 polymorphism and/or ICAM-1 rs1799969 polymorphism; and (b) indicating that the subject is more likely to respond to treatment with bevacizumab and fluoropyrimidine-based chemotherapy and/or likely to have a longer overall survival, if at least one of VEGF-A rs699947 polymorphism and ICAM-1 rs1799969 polymorphism is present. In such an instance, the subject will have the polymorphism VEGF-A rs699947 polymorphism and ICAM-1 rs1799969 polymorphism.

For example, a subject having the VEGF-A rs699947 polymorphism will have an OS of 33 to 71 months, 33.3 to 70.7 months, 40 to 65 months, 45 to 60 months, or 50 to 55 months, if treated with bevacizumab and fluoropyrimidine-based chemotherapy. For example, a subject having the VEGF-A rs699947 polymorphism will have an OS of 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, if treated with bevacizumab and fluoropyrimidine-based chemotherapy.

For example, a subject having the ICAM-1 rs1799969 polymorphism will have an OS of 34 to 63 months, 34.5 to 62.8 months, 40 to 60 months, 45 to 55 months, or 50 to 55 months, if treated with bevacizumab and fluoropyrimidine-based chemotherapy. For example, a subject having the ICAM-1 rs1799969 polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, if treated with bevacizumab and fluoropyrimidine-based chemotherapy.

The invention further provides an indication that the subject is less likely to respond to treatment with bevacizumab and fluoropyrimidine-based chemotherapy and/or less likely to have a longer overall survival, if VEGF-A rs699947 polymorphism and ICAM-1 rs1799969 polymorphism are not present in the subject's sample. In such an instance, the subject will have the wild-type genotype VEGF-A rs699947 (allele C/C) and ICAM-1 rs1799969 (allele G/G).

Identification of the Polymorphism

The sample is obtained from a subject diagnosed with mCRC (i.e. metastatic carcinoma of the colon or rectum). In preferred embodiments, the subject is an adult.

The sample may be a whole-blood, serum blood, peripheral blood leukocytes, or saliva. Other samples from which genomic DNA can be extracted are known to those skilled in the art.

The presence or absence of the allele can be identified by standard methods known in the art for detecting polymorphisms. For example, PCR, PCR-RFLP, direct sequencing, TaqMan and/or next generation signalling.

A general method for identifying a polymorphism is:
(a) Collecting a whole blood sample
(b) Separating the blood and isolating the serum sample
(c) Extracting the genomic DNA from blood leukocytes
(d) Measuring the DNA concentration
(e) Analysing the genomic variant by PCR and purifying the PCR products
(f) Carrying our DNA sequence analysis on the purified PRC products

In an alternative embodiment, a method for identifying a polymorphism is adding a label that binds to the VEGF-A rs699947 polymorphism and/or a label that binds to ICAM-1 rs1799969 polymorphism and detecting one or both of the bound labels.

In an alternative embodiment, a method for identifying a polymorphism is adding a label that binds to the VEGF-A rs699947 polymorphism and/or ICAM-1 rs1799969 polymorphism and detecting the bound label.

In some embodiments of the invention, the subject has already been identified as having the VEGF-A rs699947 polymorphism and/or ICAM-1 rs1799969 polymorphism.

Bevacizumab and Fluoropyrimidine-Based Chemotherapy

The bevacizumab and fluoropyrimidine-based chemotherapy may be a first-line treatment for mCRC. The bevacizumab and fluoropyrimidine-based chemotherapy may be administered as part of standard chemotherapy regimen as known in the art.

The fluoropyrimidine-based chemotherapy can be selected from the group consisting of 5-fluorouracil/leucovorin/irinotecan (BEV-FOLFIRI), 5-fluorouracil/leucovorin/oxaliplatin (BEV-FOLFOX), capecitabine/irinotecan (BEV-CapIRI),capecitabine/oxaliplatin (BEV-CapOX), or 5-fluorouracil/leucovorin/oxaliplatin/irinotecan (FOLFOXIRI). Each fluoropyrimidine-based chemotherapeutic option used in combination with bevacizumab is considered clinically equivalent with the other options both in the USA (NCCN) and in Europe (ESMO); see Cutsem et al. Ann Oncol. 2016 August; 27(8):1386-422 (https://www.ncbi.nlm.nih.gov/pubmed?term=27380959), and the NCCN Guidelines for colorectal cancer (https://www.nccn.org/professionals/physician_gls/default.aspx).

The bevacizumab and fluoropyrimidine-based chemotherapy can be administered by any appropriate mode of administration know the skilled person. For example, the mode of administration may be parenteral administration, such as intramuscular, subcutaneous and intravenous. The parenteral administration may be a bolus dose or an infusion over a set period of time.

The bevacizumab can be administered at a dose of: from 2 to 12.5 mg/kg, 4 to 10 mg/kg, or, 5 to 7.5 mg/kg; or 2, 2.5, 3, 3.5, 4, 4.5, 5, 5.5, 6, 6.5, 7, 7.5, 8, 8.5, 9, 9.5, 10, 10.5, 11, 11.5, 12, 12.5 mg/kg per body weight, and can be administered every week, ever 2 weeks, every 3, weeks, every 4 weeks. The treatment cycle can be repeated 2, 3, 4, 5, 6, 7, 8, 9, 10, 11, 12,13, 14, 15, 16, 17, 18, 19, 20, 21, 22, 23, 24, 25, 26, 27, 28, 29 or, 30 or more times, i.e. there can be 2, 3, 4, 5, 6, 7, 8, 9, 10, 11, 12, 13, 14, 15, 16, 17, 18, 19, 20, 21, 22, 23, 24, 25, 26, 27, 28, 29 or, 30 or more treatment cycles of bevacizumab and fluoropyrimidine-based chemotherapy.

In some embodiments, the bevacizumab is administered as an intravenous infusion at a dose of 5 mg/kg once every 2 weeks in combination with the fluoropyrimidine-based chemotherapy. In some embodiments, the bevacizumab is administered as an intravenous infusion at a dose of 5 mg/kg once every 2 weeks in combination BEV-FOLFIRI or BEV-FOLFOX.

In some embodiments, the bevacizumab is administered as an intravenous infusion at a dose of 7.5 mg/kg once every 3 weeks in combination with in 3-week cycles in combination with the fluoropyrimidine-based chemotherapy. In some embodiments, the bevacizumab is administered as an intravenous infusion at a dose of 7.5 mg/kg once every 3 weeks in combination with in 3-week cycles in combination with BEV-CapIRI or BEV-CapOX.

An intravenous infusion of bevacizumab or a bevacizumab biosimilar at a dose of 7.5 mg/kg once every 3 weeks is considered to be clinically equivalent to an intravenous infusion at a dose of 5 mg/kg once every 2 weeks in combination with the fluoropyrimidine-based chemotherapy.

Intravenous administration of bevacizumab and fluoropyrimidine-based chemotherapy allows for rapid entry into the body's circulation, where it is carried throughout the body in the blood stream. Intravenous administration offers a rapid absorption time and flexibility with drug dosing. Doses can be given as an intravenous infusion lasting from a few minutes to a few hours. In particular, doses can be given as an intravenous infusion lasting from 30 to 90 minutes. Portable pumps allow medication to be given at a slow continuous rate allowing for ongoing intravenous infusion of bevacizumab and fluoropyrimidine-based chemotherapy.

The first-line treatment may be followed by a maintenance treatment. The maintenance treatment may be selected from the list of Bevacizumab-mFOLFOX, Bevacizumab-FOLFIRI, Bevacizumab-CapIRI, Bevacizumab-De Gramont (flurouracil in combination with leucovorin or calcium folinate), Bevacizumab-Capecitabine, and/or Bevacizumab monotherapy.

For example, following 6 treatment cycles of FOLFOX/FOLFIRI or 4 treatment cycles of CapeOx/CapIri, and based on response, patients may continue maintenance treatment. The selection of maintenance treatment should be individualised and take into account the following factors:

1. Tolerability, side effects

2. Socioeconomic, logistical issues

3. Volume of disease, histology, grading, number of metastases etc.

All in all, there is no clear evidence for the duration of treatment and the best option of treatment regimens. The available option are:

1. Single agent bevacizumab

2. Bevacizumab in combination with fluropyrimidines only

3. Continuation of the initial bevacizumab with fluoropyrimidine based chemotherapy, wherein the fluoropyrimidine based chemotherapy is 5-fluorouracil/leucovorin/irinotecan (BEV-FOLFIRI), 5-fluorouracil/leucovorin/oxaliplatin (BEV-FOLFOX), capecitabine/irinotecan (BEV-CapIRI),capecitabine/oxaliplatin (BEV-CapOX), or 5-fluorouracil/leucovorin/oxaliplatin/irinotecan (FOLFOXIRI).

For example, a subject administered an initial treatment of 2-weekly bevacizumab plus fluoropyrimidine based chemotherapy, may be administered bevacizumab every 3 weeks (7.5 mg/kg) as maintenance treatment. As part of the maintenance dose, the bevacizumab can be administered at a dose of: from 2 to 12.5 mg/kg, 4 to 10 mg/kg, or, 5 to 7.5 mg/kg; or 2, 2.5, 3, 3.5, 4, 4.5, 5, 5.5, 6, 6.5, 7, 7.5, 8, 8.5, 9, 9.5, 10, 10.5, 11, 11.5, 12, 12.5 mg/kg per body weight, and can be administered every week, ever 2 weeks, every 3, weeks, every 4 weeks. The maintenance treatment cycle can be repeated 2, 3, 4, 5, 6, 7, 8, 9, 10, 11, 12,13, 14, 15, 16, 17, 18, 19, 20, 21, 22, 23, 24, 25, 26, 27, 28, 29 or, 30 or more times, i.e. there can be 2, 3, 4, 5, 6, 7, 8, 9, 10, 11, 12, 13, 14, 15, 16, 17, 18, 19, 20, 21, 22, 23, 24, 25, 26, 27, 28, 29 or, 30 or more maintenance treatment cycles of bevacizumab alone, with fluoropyrimidine only, or with a fluoropyrimidine-based chemotherapy.

The invention provides a method of selecting a treatment for a subject's mCRC, the method comprising the steps of: (a) identifying the presence or absence of the VEGF-A rs699947 polymorphism (allele A/A) and/or ICAM-1 rs1799969 polymorphism (allele G/A) in a sample obtained from the subject; and (b) selecting the treatment of a bevacizumab and fluropyrimidine-based chemotherapy, if at least one of VEGF-A rs699947 polymorphism (allele A/A) and ICAM-1 rs1799969 polymorphism (allele G/A) is present.

The invention further provides a method of selecting a treatment for a subject's mCRC, the method comprising the steps of: (a) identifying the presence or absence of the VEGF-A rs699947 polymorphism (allele A/A) and/or ICAM-1 rs1799969 polymorphism (allele G/A) in a sample obtained from the subject; and (b) selecting the treatment of fluoropyrimidine-based chemotherapy only, if VEGF-A rs699947 polymorphism (allele A/A) and ICAM-1 rs1799969 polymorphism (allele G/A) are not present in the subject's sample. In such an instance, the subject will have the wild-type genotype VEGF-A rs699947 (allele C/C) and wild-type genotype ICAM-1 rs1799969 (allele G/G).

The invention further provides a method of treating a subject's mCRC, the method comprising the steps of: (a) identifying the presence or absence of the VEGF-A rs699947 polymorphism (allele A/A) and/or ICAM-1 rs1799969 polymorphism (allele G/A) in a sample obtained from the subject; and (b) administering bevacizumab and fluropyrimidine-based chemotherapy, if at least one of VEGF-A rs699947 polymorphism (allele A/A) and ICAM-1 rs1799969 polymorphism (allele G/A) is present.

The invention provides a combination of bevacizumab and a fluoropyrimidine-based chemotherapy for use in a method of treating a subject's mCRC, the method comprising the steps of: (a) identifying the presence or absence of the VEGF-A rs699947 polymorphism (allele A/A) and/or ICAM-1 rs1799969 polymorphism (allele G/A) in a sample obtained from the subject; and (b) administering bevacizumab and fluropyrimidine-based chemotherapy, if at least one of VEGF-A rs699947 polymorphism (allele A/A) and ICAM-1 rs1799969 polymorphism (allele G/A) is present.

The invention also provides a method of treating mCRC in a subject having the VEGF-A rs699947 polymorphism and/or ICAM-1 rs1799969 polymorphism, the method comprising administering bevacizumab and fluoropyrimidine-based chemotherapy.

The invention further comprises a combination of bevacizumab and a fluoropyrimidine-based chemotherapy for use in a method of treating a subject's mCRC, wherein the subject has the VEGF-A rs699947 polymorphism and/or ICAM-1 rs1799969 polymorphism.

Reference to bevacizumab includes reference to Avastin® and equally applies to biosimilars of the reference medicine/reference product bevacizumab (Avastin®) as approved for a marketing authorisation by a regulatory authority such as the European Medicines Agency (EMA), the United States Food and Drug Administration (FDA), or the like. Hence, reference to the combination of bevacizumab and fluoropyrimidine-based chemotherapy applies to biosimilars of the reference medicine/reference product bevacizumab (Avastin®) and a fluoropyrimidine-based chemotherapy.

Also provided as part of the invention is a method of treating cancer a subject with metastatic colorectal cancer (mCRC) comprising or consisting of the steps of administering bevacizumab and fluoropyrimidine-based chemotherapy to a subject; and identifying the presence or absence of the VEGF-A rs699947 polymorphism and/or ICAM-1 rs1799969 polymorphism in a sample obtained from the subject, or identifying a subject who has been identified as having the VEGF-A rs699947 polymorphism and/or ICAM-1 rs1799969 polymorphism.

For a subject having the VEGF-A rs699947 polymorphism, the outcome of the subject will be an OS of 33 to 71 months, 33.3 to 70.7 months, 40 to 65 months, 45 to 60 months, or 50 to 55 months, when treated with bevacizumab and fluoropyrimidine-based chemotherapy. For a subject having the VEGF-A rs699947 polymorphism, the outcome of the subject will be an OS of 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 67, 68, 69, 70, or 71 months, when treated with bevacizumab and fluoropyrimidine-based chemotherapy. For a subject having the ICAM-1 rs1799969 polymorphism, the outcome of the subject will be an OS of 34 to 63 months, 34.5 to 62.8 months, 40 to 60 months, 45 to 55 months, or 50 to 55 months, when treated with bevacizumab and fluoropyrimidine-based chemotherapy. For a subject having the ICAM-1 rs1799969 polymorphism, the outcome of the subject will be an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, when treated with bevacizumab and fluoropyrimidine-based chemotherapy.

Also provided as part of the invention is a method for treating metastatic colorectal cancer in a subject, the method comprising administering a combination of bevacizumab and a fluoropyrimidine-based chemotherapy to a subject known to have the ICAM-1 rs1799969 polymorphism.

Also provided as part of the invention is a method for administering a combination of bevacizumab and a fluoropyrimidine-based chemotherapy to a subject having metastatic colorectal cancer, the method comprising identifying the presence or absence of the ICAM-1 rs1799969 polymorphism in a sample obtained from the subject and administering the combination of bevacizumab and a fluoropyrimidine-based chemotherapy to the subject having the ICAM-1 rs1799969 polymorphism.

Also provided as part of the invention is a method for treating metastatic colorectal cancer in a subject, the method comprising administering a combination of bevacizumab and a fluoropyrimidine-based chemotherapy to a subject known to have the VEGF-A rs699947 polymorphism.

Also provided as part of the invention is a method for administering a combination of bevacizumab and a fluoropyrimidine-based chemotherapy to a subject having metastatic colorectal cancer, the method comprising identifying the presence or absence of the VEGF-A rs699947 polymorphism in a sample obtained from the subject and administering the combination of bevacizumab and a fluoropyrimidine-based chemotherapy to the subject having the VEGF-A rs699947 polymorphism.

In the methods described herein, the combination of bevacizumab and a fluoropyrimidine-based chemotherapy is administered as first-line treatment or a second-line treatment.

In the methods described herein, the step of identifying comprises detecting the polymorphism by a method comprising one or more of PCR, PCR-RFLP, direct sequencing, TaqMan and/or next generation sequencing.

Bevacizumab and biosimilars inhibit the formation of new blood cells (angiogenesis) by specifically recognizing and binding to vascular VEGF-A, thereby treating mCRC. Biosimilars are approved according to the same standards of pharmaceutical quality, safety and efficacy that apply to all biological medicines. Biosimilars have clinical equivalence and no clinically meaningful differences to the reference medicine/reference product bevacizumab (Avastin®). Therefore, subjects having the VEGF-A rs699947 polymorphism and/or ICAM-1 rs1799969 polymorphism will respond to treatments including biosimilars of the reference medicine/reference product bevacizumab (Avastin®), in the same manner as treatments including of the reference medicine/reference product bevacizumab (Avastin®). Bevacizumab biosimilars are known in the art, for example but not limited to: Mvasi (ABP 215) or (bevacizumab-awwb) (Amgen) and Zirabev (Pfizer). Mvasi (ABP 215) is a recombinant IgG1 humanised monoclonal antibody produced from Chinese hamster ovary cells. Mvasi (ABP 215) has the same primary structure as bevacizumab. Mvasi (ABP 215) binds to vascular endothelial growth factor (VEGF), thereby inhibiting the binding of VEGF to its receptors on the surface of endothelial cells. Neutralising the biological activity of VEGF regresses the vascularisation of tumours, normalises remaining tumour vasculature, and inhibits the formation

Zirabev is a recombinant IgG1 humanised monoclonal antibody produced from Chinese hamster ovary cells. Zirabev binds to vascular endothelial growth factor (VEGF), thereby inhibiting the binding of VEGF to its receptors on the surface of endothelial cells. Neutralising the biological activity of VEGF regresses the vascularisation of tumours, normalises remaining tumour vasculature, and inhibits the formation of new tumour vasculature, thereby inhibiting tumour growth.

A number of bevacizumab biosimilars are also being developed, such as but not limited to BCD-021 (Biocad, Russia); FKB238 (AstraZeneca/Fujifilm Kyowa Kirin Biologics, USA/Japan); BCD500 (BIOCND, South Korea); Krabeva (Biocon, India); BI 695502 (Boehringer Ingelheim, Germany); CT-P16 (Celltrion, South Korea); CHS-5217 (Coherus, USA); DRZ_BZ (Dr Reddy's Laboratories, India); Cizumab (Hetero (Lupin), India); Bevax (mAbxience, Spain (Argentina)); ONS-1045 (OncobiologicsNiropro, USA); PF-06439535 (Pfizer, USA); HD204 (Prestige Biopharma, Singapore); Bevacirel (Reliance Life Sciences/Lupin, India); and SB8 (Samsung Bioepis (Biogen/Samsung)/Merck, South Korea/USA); these compounds will be bevacizumab biosimilars in accordance with the invention if they are approved as a biosimilar of bevacizumab by an appropriate regulatory authority such as the European Medicines Agency (EMA), the United States Food and Drug Administration (FDA), or the like.

Based on the fact that the biosimilars of the reference medicine/reference product bevacizumab (Avastin®) have high structural and functional similarity to bevacizumab, the data provided herein can be extrapolated to biosimilars of the reference medicine/reference product bevacizumab (Avastin®). In particular, biosimilars bind to and inhibit the biologic activity of human vascular endothelial growth factor (VEGF-A). The interaction of VEGF with its receptors leads to endothelial cell proliferation and new blood vessel formation in in vitro models of angiogenesis. Biosimilars bind to circulating VEGF-A and block VEFG-A from binding to is receptors (VEGFR-1 and VEGFR-2) on the surface of endothelial cells, which results in the inhibition of tumour angiogenesis, growth and metastases.

Biosimilars are highly similar to the reference medicine/reference product bevacizumab (Avastin®) in terms of structure, biological activity and efficacy, safety and immunogenicity profile. These biosimilars must have clinical equivalence to the reference medicine/reference product bevacizumab (Avastin®) and no clinically meaningful differences can exist between the biosimilar and the reference medicine/reference product bevacizumab (Avastin®). In particular, there are no clinically meaningful differences between the biosimilar and the bevacizumab (the reference medicine/reference product) in terms of safety, quality and efficacy.

In some embodiments, the bevacizumab is the reference medicine/reference product bevacizumab (Avastin®).

In some embodiments, the bevacizumab is a biosimilar of is the reference medicine/reference product bevacizumab (Avastin®). In some embodiments, the bevacizumab is a biosimilar of is the reference medicine/reference product bevacizumab (Avastin®) selected from the list consisting of: Mvasi (ABP 215) (Amgen) and Zirabev (Pfizer). These biosimilars have clinical equivalence and no clinically meaningful differences to the reference medicine/reference product bevacizumab (Avastin®).

In some embodiments, the bevacizumab is a biosimilar of is the reference medicine/reference product bevacizumab (Avastin®) selected from the list consisting of: BCD-021 (Biocad, Russia); FKB238 (AstraZeneca/Fujifilm Kyowa Kirin Biologics, USA/Japan); BCD500 (BIOCND, South Korea); Krabeva (Biocon, India); BI 695502 (Boehringer Ingelheim, Germany); CT-P16 (Celltrion, South Korea); CHS-5217 (Coherus, USA); DRZ_BZ (Dr Reddy's Laboratories, India); Cizumab (Hetero (Lupin), India); Bevax (mAbxience, Spain (Argentina)); ONS-1045 (OncobiologicsNiropro, USA); PF-06439535 (Pfizer, USA); HD204 (Prestige Biopharma, Singapore); Bevacirel (Reliance Life Sciences/Lupin, India); and SB8 (Samsung Bioepis (Biogen/Samsung)/Merck, South Korea/USA); when approved as a biosimilar of the reference medicine/reference product bevacizumab (Avastin®) by an appropriate regulatory authority such as the European Medicines Agency (EMA), the United States Food and Drug Administration (FDA), or the like.

### EXAMPLES

Patients and Treatment

A confidential, observational, real-world study, was conducted in the Department of Oncology, University Hospital of Patras, Greece, between July 2012 and July 2018. Patients were 18 years of age or older, had ECOG performance status 0-2 and histopathologically confirmed mCRC. All patients received first-line treatment with bevacizumab in combination with a fluoropyrimidine-based chemotherapy.

Bevacizumab (Avastin®) was administered as an intravenous infusion at a dose of 5 mg/kg once every 2 weeks in combination with 5-fluorouracil/leucovorin/irinotecan or oxaliplatin (BEV-FOLFIRI or BEV-FOLFOX, respectively); or at a dose of 7.5 mg/kg once every 3 weeks in combination with capecitabine/irinotecan or oxaliplatin (BEV-CapIRI or BEV-CapOX, respectively) in 3-week cycles. Treatment was initially administered for six (BEV-FOLFIRI, BEV-FOLFOX) or four (BEV-CapIRI, BEV-CapOX) cycles; patients who responded to treatment, continued with bevacizumab-based maintenance treatment. Radiographic evaluation was performed every 8-12 weeks or when clinically indicated. The response was evaluated according to RECIST criteria version

Maintenance treatment was decided by the treating physicians. The maintenance treatments were selected from: Bevacizumab-mFOLFOX, Bevacizumab-FOLFIRI, Bevacizumab-CapIRI, Bevacizumab-De Gramont, Bevacizumab-Capecitabine, and Bevacizumab monotherapy. The endpoints of progression-free survival (PFS) and overall survival (OS). The study was conducted in accordance with the Declaration of Helsinki and the International Conference on Harmonisation (ICH) Good Clinical Practice. Approval was obtained by the Hospital's Ethics Committee. Prior to study enrolment, all patients provided signed informed consent.

Pharmacogenetic Analysis

Whole-blood samples were collected at baseline in order to analyse the VEGF-A (r52010963, rs1570360, rs699947) and ICAM-1 (rs5498, rs1799969) SNPs. Blood samples were collected in serum separator tubes and were allowed to clot for 30 min. After centrifugation at 1000×g for 20 min, the serum was removed and stored in aliquots at ≤−20° C. until analysis. Genomic DNA was extracted from peripheral blood leukocytes from patients using the Gentra Puregene Blood kit (QIAGEN) (see DNA sequencing by capillary electrophoresis. Applied Biosystems chemistry guide, 2nd edition; https://tools.thermofisher.com/content/sfs/manuals/cms_041003.pdf; incorporated herein by reference). DNA concentrations were determined by measuring the optical density at 260 nm with a UV-Vis spectrophotometer (NanoDrop 2000, Thermo Fisher Scientific). DNA purity, which is indicated by the ratio of optical density at 260 and 280 nm, was 1.7-1.9. VEGF-A (rs699947, rs1570360, and rs2010963) and ICAM-1 (rs1799969, rs5498) genomic variants were analysed by polymerase chain reaction (PCR) according to the KAPA2G Fast HotStart protocol (KAPABIOSYSTEMS, MA, USA). PCR products were separated on 1% w/v agarose gels stained with Midori Green and were purified using the PCR and DNA Fragment Purification kit (Dongsheng Biotech, DNA purity 1.7-1.9). Following purification, samples were subjected to direct DNA sequence analysis on an ABI Prim 3130xI DNA Analyser (Applied Biosystems) using the Big Dye® Terminator v3.1 Cycle Sequencing Kit (Applied Biosystems, CA, USA), according to the manufacturer's instructions.

KRAS, NRAS (exons 2, 3, 4) and BRAF (exons 11 and 15) mutation status was determined in genomic DNA extracted from formalin-fixed, paraffin-embedded tissue samples from the patients with QIAmp DNA FFPE tissue kit (Qiagen/MagCore Genomic DNA FFPE One-Step Kit, RBC Bioscience). Mutations in exons 2, 3, 4 of the KRAS and NRAS genes, and in exons 11 and 15 of BRAF gene were detected with the method of targeted realignment (Ion AmpliSeq□Panel, Thermo Fisher Scientific). Next generation sequencing platform Ion proton (Thermo Fisher Scientific) was used for sequencing. The method's detection limit was 2-5% (mutant/wild-type alleles).

Statistics

All categorical data including therapy methods and polymorphisms were tabulated and presented as frequencies and counts (see Table 1). Statistical significance was set at p<0.05.

For the time-to-event analyses, we used the Kaplan-Meier estimate, and all lost to follow-up cases were censored up to the most recent available time-point. The OS and PFS median estimates are presented with 95% confidence intervals. The log-rank test was used for the comparison of the OS and PFS distributions for different polymorphisms, while the effects of age and sex were investigated with multiple analysis using Cox regression. In terms of the sample size calculation, there were feasibility limitations in recruitment and, therefore, the investigators calculated the effect size that this study could capture for the pre-determined number of patients. At a power level of 80% and alpha set as 0.05, for 46 patients with 60 months of follow up and 1 year of recruitment we can detect at least 1.5 years of difference in the median of PFS and OS between different polymorphisms. The Kaplan Meier graphs were generated using GraphPad Prism version 7 for Windows, GraphPad Software, La Jolla Calif. USA. The analyses were performed using SAS version 9.4, SAS Institute Inc. 2015. SAS/IML® 14.1 User's Guide. Cary, N.C.: SAS Institute Inc.

Results

Patient Population

A total of 46 consecutive patients with mCRC were enrolled in the study. Overall, patients had a mean age of 64.5 years (range 31 to 86) and were predominantly male (28/46; 61%). The most commonly used initial treatment was BEV-FOLFOX (46%) and the most common maintenance treatment was bevacizumab monotherapy (30%) (Table 1). The mean number of metastatic sites was 2 (range 1 to 5).

Genotypes

Most patients had wild-type variants of VEGF-A SNPs: rs2010963 (58.7%), rs1570360 (67.4%) and rs699947 (74%). Similarly, the wild-type ICAM-1 gene rs1799969 was dominant (78.3%), but 50% of patients were heterozygous for rs5498. Fifty percent of patients had KRAS wild-type tumors, and 61.2% presented with NRAS mutations. The vast majority (81.8%) of patients had wild-type BRAF tumors.

Genotype frequencies are summarised in Table 1.

TABLE 1
Baseline patient characteristics, therapeutic regimens used and
genotype frequencies

| | Frequency (%) |
|---|---|
| Sex | |
| Female | 39% |
| Male | 61% |
| Age (years) | |
| <55 | 30% |
| 55-65 | 26% |
| >65 | 44% |
| Metastatic site | |
| 1 | 33% |
| 2 | 26% |
| >2 | 41% |
| Initial treatment | |
| Bevacizumab-mFOLFOX6 | 48% |
| Bevacizumab-FOLFIRI | 28% |
| Bevacizumab-CapOX | 4% |
| Bevacizumab-CapIRI | 20% |
| Maintenance treatment | |
| Bevacizumab-mFOLFOX | 69% |
| Bevacizumab-FOLFIRI | 10% |
| Bevacizumab-CapIRI | 20% |
| Bevacizumab-De Gramont | 24% |
| Bevacizumab-Capecitabine | 7% |
| Bevacizumab monotherapy | 30% |
| VEGF-A rs2010963 | |
| G/G | 58.7% |
| G/C | 28.3% |
| C/C | 13% |
| VEGF-A rs1570360 | |
| G/G | 67.4% |
| G/A | 21.7% |
| A/A | 10.9% |
| VEGF-A rs699947 | |
| C/C | 74% |
| C/A | N/A |
| A/A | 26% |
| ICAM-1 rs5498 | |
| A/A | 28.3% |
| A/G | 50% |
| G/G | 21.7% |
| ICAM-1 rs1799969 | |
| G/G | 78.3% |
| G/A | 21.7% |
| A/A | N/A |
| KRAS rs61764370 | |
| Wild type | 50% |
| Mutant | 50% |
| NRAS rs11554290 | |
| Wild type | 38.8% |
| Mutant | 61.2% |
| BRAF rs113488022 | |
| Wild type | 81.8% |
| Mutant | 18.2% |

NOTE:
Bevacizumab-mFOLFOX6 (bevacizumab 5 mg/kg, oxaliplatin 85 mg/m2, folinic acid 400 mg/m2, fluorouracil 400 mg/m2 bolus, fluorouracil 2400 mg/m2 over 46 hours every 2 weeks), Bevacizumab-FOLFIRI (bevacizumab 5 mg/kg, irinotecan 180 mg/m2, folinic acid 400 mg/m2, fluorouracil 400 mg/m2 bolus, fluorouracil 2400 mg/m2 over 46 hours every 2 weeks), Bevacizumab-CapOX (bevacizumab 7.5 mg/kg on d 1, oxaliplatin 130 mg/m2 on d 1, capecitabine 1000 mg/m2/12 h d 1-14 every 3 weeks), Bevacizumab-CapIRI (bevacizumab 7.5 mg/kg on d 1, irenotecan 250 mg/m2 on d 1, capecitabine 1000 mg/m2/12 h d 1-14 every 3 weeks), Bevacizumab-De Gramont (bevacizumab 5 mg/kg, folinic acid 200 mg/m2 on d 1, 2, fluorouracil 400 mg/m2 bolus on d 1, 2, fluorouracil 2400 mg/m2 over 22 hours on d 1, 2 every 2 weeks), Bevacizumab-Capecitabine (bevacizumab 7.5 mg/kg on d 1, capecitabine 1000 mg/m2/12 h d 1-14 every 3 weeks), Bevacizumab monotherapy (5 mg/kg every 2 weeks or 7.5 mg/kg every 3 weeks).

Clinical Response

After a median follow up of 60 months, 88.1% of patients progressed and 58.0% died.

For all patients, median PFS and OS were 9 and 18 months, respectively. In terms of response, complete response (CR) was achieved by one patient, partial response (PR) by (34.8%) patients, stable disease by (54.4%) patients, and progressive disease (PD) in (8.7%) patients, all at the first imaging evaluation.

TABLE 2
Response assessment by RECIST 1.1 and analysis of
polymorphisms for DCR.

| | CR, PR or | | |
|---|---|---|---|
| SNP | SD | PD | p-value |
| VEGF-A rs2010963 | | | |
| G/G | 89% | 11% | 0.380 |
| G/C | 100% | 0% | |
| C/C | 83.3% | 16.7% | |
| VEGF-A rs1570360 | | | |
| G/G | 90.3% | 9.7% | 0.410 |
| G/A | 100% | 0% | |
| A/A | 80% | 20% | |
| VEGF-A rs699947 | | | |
| C/C | 85% | 15% | 0.199 |
| C/A | N/A | N/A | |
| A/A | 100% | 0% | |
| ICAM-1 rs5498 | | | |
| A/A | 92.3% | 7.7% | 0.477 |
| A/G | 95.5% | 4.5% | |
| G/G | 80% | 20% | |
| ICAM-1 rs1799969 | | | |
| G/G | 89% | 11% | 0.295 |
| G/A | 100% | 0% | |
| A/A | N/A | N/A | |
| KRAS rs61764370 | | | |
| Wild type | 83.3% | 16.7% | 0.537 |
| Mutant | 91.7% | 8.3% | |
| NRAS rs11554290 | | | |
| Wild type | 100% | 0% | 0.601 |
| Mutant | 71.4% | 28.6% | |
| BRAF rs113488022 | | | |
| Wild type | 100% | 0% | 0.026 |
| Mutant | 50% | 50% | |

NOTE:
DCR: Disease Control Rate, CR: Complete Response, PR: Partial Response, SD: Stable Disease, PD: Progressive Disease.

Impact of Gene Polymorphisms on Treatment Efficacy

The presence of any polymorphism in the VEGF-A, ICAM-1, KRAS and NRAS genes did not affect DCRs (Table 2). In contrast, BRAF mutation status was significantly (p=0.026) associated with resistance to treatment, as 50% of patients with BRAF mutant tumours had PD at first imaging evaluation. On the other hand, disease control was achieved in 100% of the patients with tumours carrying wild-type BRAF.

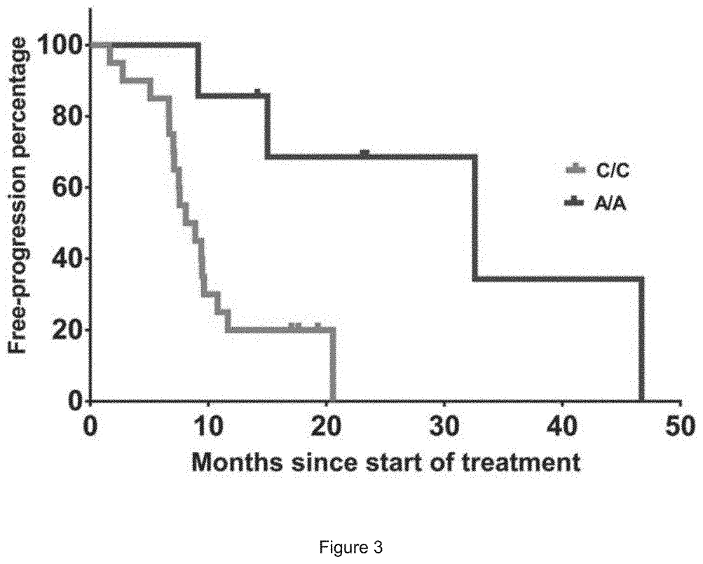

Patients homozygous for VEGF-A rs699947 A/A had significantly (p=0.006) prolonged PFS of 32.6 months compared with 8.1 months for the carriers of the wild-type C/C variant (Table 3 and FIG. 3). No other statistically significant were found between VEGF-A rs1570360 and rs2010963, ICAM-1 rs5498 and rs1799969, KRAS, NRAS, and BRAF gene variants and PFS (Table 3).

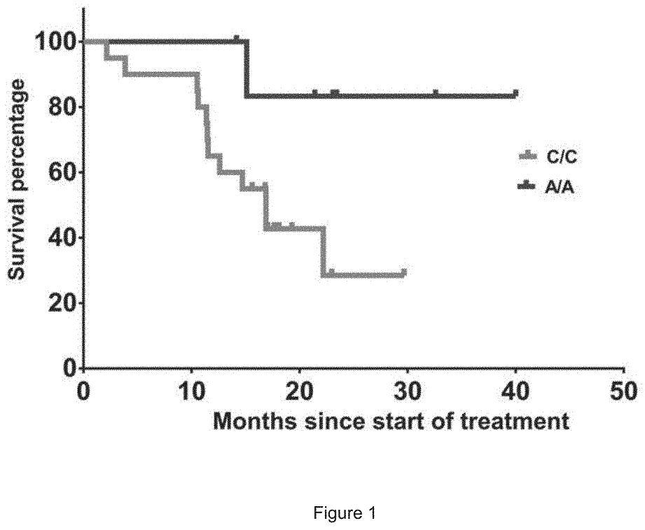

Unexpectedly, OS was significantly (p=0.043) prolonged to 59.4 months in carriers of VEGF-A rs699947 A/A compared with 16.9 months for carriers of the wild-type C/C variant (Table 3 and FIG. 1). Hence, patients carrying the VEGF-A allele rs699947 A/A are significantly associated with a longer OS and more likely to respond to the treatment of bevacizumab and fluoropyrimidine-based chemotherapy.

Patients heterozygous for VEGF-A rs1570360 G/A had longer OS compared with carriers of the G/G or A/A variants (43.2, 27.8 and 39.2 months, respectively), albeit that the difference did not meet statistical significance (p=0.074). None of the variants of the VEGF-A rs2010963 were associated with OS (p=0.811).

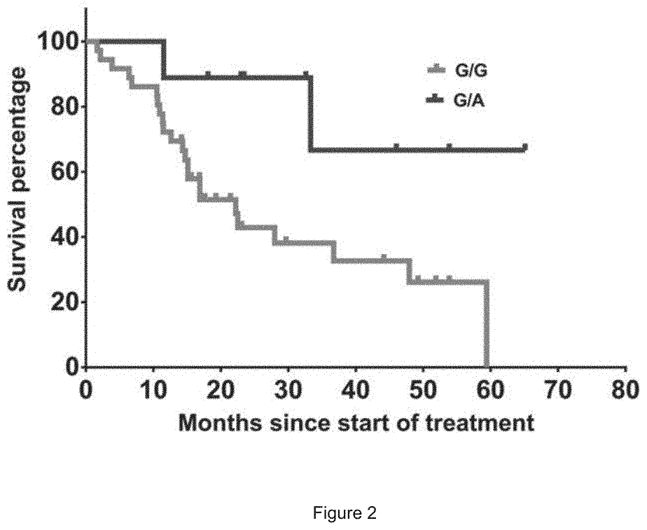

Unexpectedly, patients carrying the ICAM-1 allele rs1799969 G/A attained median OS of 48.7 months compared to 29.1 months in patients carrying the G/G allele (p=0.036) (Table 3 and FIG. 2). Hence, patients carrying the ICAM-1 allele rs1799969 G/A are significantly associated with a longer OS and more likely to respond to the treatment of bevacizumab and fluropyrimidine-based chemotherapy.

ICAM-1 rs5498 was not significantly associated with OS (p=0.159). Moreover, the median OS of patients with wild-type BRAF was significantly longer compared with patients with the mutant BRAF (16.7 versus 6.8 months, p=0.027). KRAS and NRAS were not significantly associated with OS (p values 0.511 and 0.374, respectively; Table 3). Age and sex did not appear to have any statistically significant association to DCR, PFS or OS in patients with mCRC who were treated with bevacizumab and fluropyrimidine-based chemotherapy.

TABLE 3
Analysis of polymorphisms for PFS and OS.

| | PFS | | | OS | | |
|---|---|---|---|---|---|---|
| | Median | | | Median | | |
| | months | | | months | | p- |
| Polymorphism | (95% CI) | | p-value | (95% CI) | | value |
| VEGF-A | | | | | | |
| rs2010963 | | | | | | |
| G/G | 7.6 | (4.8-10.4) | 0.600 | 35.4 | (11.6-59.2) | 0.811 |
| G/C | 9.6 | (5.9-13.2) | | 22.5 | (21.5-47.6) | |
| C/C | 8.9 | (7.1-10.7) | | 16.9 | (15-18.8) | |
| VEGF-A | | | | | | |
| rs1570360 | | | | | | |
| G/G | 8.9 | (6.9-10.9) | 0.554 | 27.8 | (20-35.6) | 0.074 |
| G/A | 12.6 | (1.6-23.6) | | 43.2 | (33.2-53.2) | |
| A/A | 9.3 | (3.7-18.9) | | 39.2 | (12.4-65.9) | |
| VEGF-A | | | | | | |
| rs699947 | | | | | | |
| C/C | 8.1 | (5.3-10.9) | 0.006 | 16.9 | (12.8.-20.9) | 0.043 |
| C/A | N/A | | | N/A | | |
| A/A | 32.6 | (13.2-44.9) | | 59.4 | (33.3-70.7) | |
| ICAM-1 | | | | | | |
| rs5498 | | | | | | |
| A/A | 8.9 | (6.6-11.2) | 0.700 | 16.9 | (12.6-21.2) | 0.159 |
| A/G | 8.1 | (4.8-11.4) | | 35.4 | (11.2-59.5) | |
| G/G | 9.4 | (4.6-14.2) | | 47.9 | (26.5-85.7) | |
| ICAM-1 | | | | | | |
| rs1799969 | | | | | | |
| G/G | 9.1 | (7-11.2) | 0.938 | 29.1 | (21.4-36.9) | 0.036 |
| G/A | 8.1 | (3.5-12.7) | | 48.7 | (34.5-62.8) | |
| A/A | N/A | | | N/A | | |
| KRAS | | | | | | |
| rs61764370 | | | | | | |
| Wild type | 8.9 | (3.9-13.8) | | 43.5 | (29-57.7) | |
| Mutant | 8.1 | (5.6-10.7) | 0.880 | 20.8 | (13.9-27.7) | 0.511 |
| NRAS | | | | | | |
| rs11554290 | | | | | | |
| Wild type | 8.9 | (6.2-11.6) | | 25 | (19.2-30.9) | |
| Mutant | 8.1 | (5.3-10.9) | 0.783 | 33.7 | (19.2-30.9) | 0.374 |
| BRAF | | | | | | |
| rs113488022 | | | | | | |
| Wild type | 7.1 | (6.8-7.4) | | 16.7 | (14.3-19.1) | |
| Mutant | 5.6 | (3.9-13.4) | 0.466 | 6.8 | (4.6-15.9) | 0.027 |

NOTE:
PFS: Progression Free Survival, OS: Overall Survival.

Discussion

The present inventors results demonstrate the statistically significant association between specific polymorphisms in VEGF-dependent and non-VEGF-dependent genes with improved clinical outcomes in patients with mCRC receiving bevacizumab and fluoropyrimidine-based chemotherapy treatment. Patients homozygous for VEGF-A rs699947 A/A had significantly prolonged PFS and OS as compared to carriers of the wild-type C/C variant (PFS:32.6 versus 8.1 months and OS:59.4 versus 16.9 months, respectively). This is the first time that a statistically significant association is observed between VEGF-A rs699947 A/A and OS in mCRC patients treated with bevacizumab in combination with fluoropyrimidine-based chemotherapy. This provides a validated biomarker that can optimise the management of mCRC by selecting the appropriate treatment. One of the main benefits of the study is a 60 month (i.e. 5 year) follow-up period, which provides better long term survival data.

In addition, increasing OS trends were noticed in carriers of VEGF-A rs1570360 G/A and A/A of 43.2 months and 39.2 months respectively versus 27.8 months of the corresponding G/G variant.

There are reports where no correlation could be identified between VEGF-A polymorphisms (including rs699947) and OS in patients with mCRC treated with bevacizumab (see Cui et al., Oncotarget 2017, 8(62), pages 105472-104478; incorporated herein by reference). The disparity between our results and those of Cui et al. may be due to the different study designs and/or statistical analysis carried out. Moreover, Cui et al. notes that their results are limited by the relatively small sample size and the single ethnicity.

Genotyping in 173 mCRC patients treated with bevacizumab in combination with FOLFIRI or CapIRI, showed that rs1570360 G/G was significantly associated with inferior OS compared to G/A.

In our study, OS was significantly associated with the ICAM-1 rs1799969 G/A variant as compared with the G/G variant (48.7 vs 29.1 months, respectively; p=0.036). This is the first time that a significant association is observed between the ICAM-1 gene and OS in mCRC patients treated with bevacizumab in combination with fluoropyrimidine-based chemotherapy.

The present inventor's results indicate that mCRC carriers of ICAM-1 rs1799969 G/A allele have a benefit of 19.6 months of survival compared to carriers of the wild-type G/G allele when treated with bevacizumab in combination with fluoropyrimidine-based chemotherapy. This provides a validated biomarker that can optimise the management of mCRC by selecting the appropriate treatment.

Furthermore, it is important to clarify that this was a pragmatic study and therefore the combinations of chemotherapy were not homogenous for all patients. From a real-world perspective, this represents the handling of the patient in the clinic. Subsequently identifying SNPs of great importance can, in fact, have an impact on delaying disease progression hence prolonging the survival and quality of life of the patient.

In conclusion, the present inventors have identified major angiogenesis and intracellular signalling pathway polymorphisms, VEGF-A rs699947 A/A and ICAM-1 rs1799969 G/A, as predictors of response to first-line bevacizumab and fluoropyrimidine based chemotherapy treatment in mCRC.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. A method for determining whether a subject with metastatic colorectal cancer (mCRC) is likely to respond to the treatment and/or for predicting overall survival (OS) of the subject when treated with bevacizumab and fluoropyrimidine-based chemotherapy or a bevacizumab biosimilar and fluoropyrimidine-based chemotherapy, comprising or consisting of the steps of:
(a) identifying the presence or absence of the ICAM-1 rs1799969 G/A allele ymoiphism and/or VEGF-A rs699947 A/A allele polymorphism in a sample obtained from the subject; and
(b) indicating that the subject is more likely to respond to treatment with bevacizumab and fluoropyrimidine-based chemotherapy or a bevacizumab biosimilar and fluoropyrimidine-based chemotherapy, and/or likely to have a longer overall survival when treated with bevacizumab and fluoropyrimidine-based chemotherapy or a bevacizumab biosimilar and fluoropyrimidine-based chemotherapy, if at least one of ICAM-1 rs1799969 G/A allele polymorphism and VEGF-A rs699947 A/A allele polymorphism is present.

2. A method of selecting a treatment for a subject's metastatic colorectal cancer, the method comprising, consisting essentially of, or consisting of the steps of:
(a) identifying the presence or absence of the ICAM-1 rs1799969 G/A allele polymorphism and/or VEGF-A rs699947 A/A allele polymorphism in a sample obtained from the subject; and
(b) selecting the treatment comprising, consisting essentially of, or consisting of a bevacizumab and fluoropyrimidine-based chemotherapy or a bevacizumab biosimilar and fluoropyrimidine-based chemotherapy if at least one of ICAM-1 rs1799969 G/A allele polymorphism and. VEGF-A rs699947 A/A allele polymorphism is present.

3. A combination of bevacizumab and a fluoropyrimidine-based chemotherapy, or a bevacizumab biosimilar and fluoropyrimidine-based chemotherapy, for use in a method of treating a subject's metastatic colorectal cancer, the method comprising the steps of:
(a) identifying the presence or absence of the ICAM-1 rs1799969 G/A allele polymorphism and/or VEGF-A rs699947 A/A allele polymorphism in a sample obtained from the subject, or identifying a subject who has been identified as having the ICAM-1 rs1799969 G/A allele polymorphism and/or VEGF-A rs699947 A/A allele polymorphism; and
(b) administering bevacizumab and fluoropyrimidine-based chemotherapy or a bevacizumab biosimilar and fluoropyrimidine-based chemotherapy, if at least one of ICAM-1 rs1799969 G/A allele polymorphism and VEGF-A rs699947 A/A allele polymorphism is present.

4. A combination of bevacizumab and a fluoropyrimidine-based chemotherapy or a bevacizumab biosimilar and fluoropyrimidine-based chemotherapy, for use in a method of treating a subject's metastatic colorectal cancer, wherein the subject has the ICAM-1 rs1799969 G/A allele polymorphism and/or VEGF-A rs699947 A/A allele polymorphism.

5. The method according to claim 1, wherein the sample is a whole-blood, blood serum, peripheral blood leukocytes, or saliva.

6. The method according to claim 1, wherein the presence or absence of the allele is identified by PCR, PCR-RFLP, direct sequencing, TaqMan and/or next generation sequencing.

7. The method according to claim 1, wherein the fluoropyrimidine-based chemotherapy is 5-fluorouracil/leucovorin/irinotecan (BEV-FOLFIRI) or 5-fluorouracil/leucovorin/oxaliplatin (BEV-FOLFOX).

8. The method according to claim 1, wherein the fluoropyrimidine-based chemotherapy is capecitabine/irinotecan (BEV-CapIRI) and/or capecitabine/oxaliplatin (BEV-CapOX).

9. The method according to claim 1, wherein the bevacizumab or the bevacizumab biosimilar is administered as an intravenous infusion at a dose of 2.5 to 7.5 mg/kg once every 2 weeks in combination with the fluoropyrimidine-based chemotherapy; preferably the dose is 5 mg/kg.

10. The method according to claim 1, wherein the bevacizumab or the bevacizumab biosimilar is administered as an intravenous infusion at a dose of 5 to 10 mg/kg once every 3 weeks in combination with in 3-week cycles in combination with the fluoropyrimidine-based chemotherapy; preferably the dose is 7.5 mg/kg.

11. The method according to claim 1, wherein the subject has 1, 2, 3, 4, 5, 6, 7, 8, 9, 10, 11, 12, 13, 14, 15, 16, 17, 18, 19, 20, 21, 22, 23, 24, 25, 26, 27, 28, 29 or, 30 treatment cycles.

12. The method according to claim 1, wherein the presence of the ICAM-1 rs1799969 G/A allele polymorphism and/or VEGF-A rs699947 A/A allele polymorphism in the sample obtained from the subject, or the subject who has been identified as having the ICAM-1 rs1799969 G/A allele polymorphism and/or VEGF-A rs699947 A/A allele polymorphism, indicates that the subject is also more likely to respond to a maintenance treatment and/or likely to have a longer overall survival with a maintenance treatment.

13. The method according to claim 1, wherein the subject has 1, 2, 3, 4, 5, 6, 7, 8, 9, 10, 11 12, 13, 14, 15, 16, 17, 18, 19, 20, 21 22, 23, 24, 25, 26, 27, 28, 29 or, 30 maintenance treatment cycles.

14. The method according to claim 12, where in the maintenance treatment is Bevacizumab-mFOLFOX6, Bevacizumab-FOLFIRI, Bevacizumab-CapIRI, Bevacizumab-De Gramont, Bevacizumab-Capecitabine, and Bevacizumab monotherapy.

15. The method according to claim 1, wherein a subject having the ICAM-1 rs1799969 G/A allele polymorphism will have an OS of 34 to 63 months, 34.5 to 62.8 months, 40 to 60 months, 45 to 55 months, or 50 to 55 months, when treated with bevacizumab and fluoropyrimidine-based chemotherapy or a bevacizumab biosimilar and fluoropyrimidine-based chemotherapy; or a subject having the ICAM-1 rs1799969 G/A allele polymorphism will have an OS of 34, 34, 36, 37, 38, 39, 40, 41, 42, 43, 44, 45, 46, 47, 48, 49, 50, 51, 52, 53, 54, 55, 56, 57, 58, 59, 60, 61, 62, 63 months, when treated with bevacizumab and fluoropyrimidine-based chemotherapy or a bevacizumab biosimilar and fluoropyrimidine-based chemotherapy.

16. The method according to claim 1, wherein a subject having the VEGF-A rs699947 A/A allele polymorphism will have an OS of 33 to 71 months, 33.3 to 70.7 months, 40 to 65 months, 45 to 60 months, or 50 to 55 months, when treated with bevacizumab and fluoropyrimidine-based chemotherapy or a bevacizumab biosimilar and fluoropyrimidine-based chemotherapy; or a subject having the VEGF-A rs699947 A/A allele polymorphism will have an OS of 33, 34, 35, 36, 37, 38, 39, 40, 41, 42, 43, 44, 45, 46, 47, 48, 49, 50, 51, 52, 53, 54, 55, 56, 57, 58, 59, 60, 61 62, 63, 67, 68, 69, 70, or 71 months, when treated with bevacizumab and fluoropyrimidine-based chemotherapy or a bevacizumab biosimilar and fluoropyrimidine-based chemotherapy.

17. A method of treating cancer a subject with metastatic colorectal cancer (mCRC) comprising or consisting of the steps of:
administering bevacizumab and fluoropyrimidine-based chemotherapy or a bevacizumab biosimilar and fluoropyrimidine-based chemotherapy to a subject; and
identifying the presence or absence of the ICAM-1 rs1799969 G/A allele polymorphism and/or VEGF-A rs699947 A/A allele polymorphism in a sample obtained from the subject, or identifying a subject who has been identified as having the ICAM-1 rs1799969 G/A allele polymorphism and/or VEGF-A rs699947 A/A allele polymorphism.

18. The method according to claim 2, wherein the sample is a whole-blood, blood serum, peripheral blood leukocytes, or saliva.

19. The method according to claim 2, wherein the presence or absence of the allele is identified by PCR, PCR-RFLP, direct sequencing, TaqMan and/or next generation sequencing.

20. The method according to claim 2, wherein the fluoropyrimidine-based chemotherapy is 5-fluorouracil/leucovorin/irinotecan (BEV-FOLFIRI) or 5-fluorouracil/eucovorin/oxaliplatin (BEV-FOLFOX).
